(19) United States
(12) Patent Application Publication
(10) Pub. No.: US 2010/0249461 A1
(43) Pub. Date: Sep. 30, 2010

(54) TRIARYLAMINE DERIVATIVE

(76) Inventors: Tomohito MASAKI, Kanagawa (JP); Keizo Kimura, Kanagawa (JP); Osamu Uchida, Kanagawa (JP)

Correspondence Address: BIRCH STEWART KOLASCH & BIRCH, PO BOX 747, FALLS CHURCH, VA, 22040-0747, US

(21) Appl. No.: 12/796,433

(22) Filed: Jun. 8, 2010

Related U.S. Application Data
(62) Division of application No. 12/100,946, filed on Apr. 10, 2008.

(30) Foreign Application Priority Data
Apr. 13, 2007 (JP) 2007-106371

(51) Int. Cl. C07C 211/26

(52) U.S. Cl. 564/434

(57) ABSTRACT

A triarylamine derivative is represented by the following Formula (I). In Formula (I): R¹¹¹, R¹¹², R¹²¹, R¹²², R¹³¹ and R¹³² each independently represent a hydrogen atom, an aliphatic group or an aromatic group, provided that R¹³¹ and R¹³² are not both phenyl groups having a group represented by —NR¹⁴¹R¹⁴² at a fourth position; R¹⁴¹ and R¹⁴² each independently represent a hydrogen atom, an aliphatic group, or an aromatic group; R¹¹³, R¹²³ and R¹³³ each independently represent a substituent; n₁₁₃, n₁₂₃ and n₁₃₃ each independently represent an integer of from 0 to 4; and X⁻ represents a counteranion represented by any of the following Formulae (II) to (IV). In Formulae (II) to (IV), R²¹¹, R²¹², R³¹¹, R³¹², R⁴¹¹, R⁴¹² and R⁴¹³ each independently represent an aliphatic group, an aromatic group or a heterocyclic group.

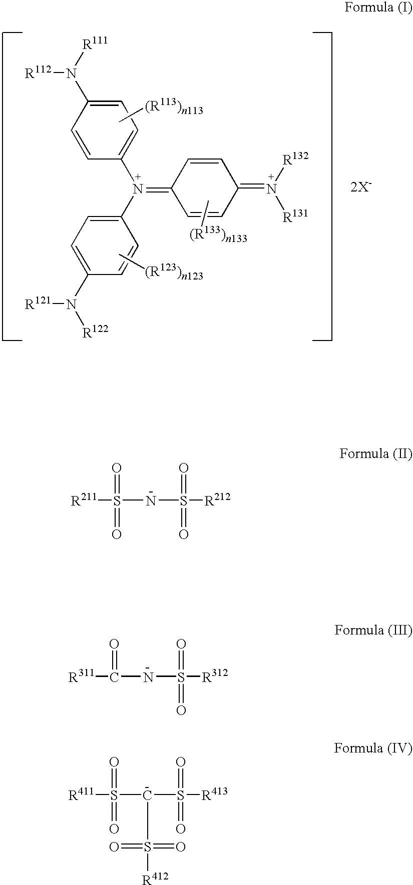

### CROSS-REFERENCE TO RELATED APPLICATION

This Application is a Divisional of co-pending application Ser. No. 12/100,946, filed on Apr. 10, 2008, which claims priority under 35 U.S.C. §119 from Japanese patent Application No. 2007-106371, filed on Apr. 13, 2007, the entire contents of which are hereby incorporated by reference in their entirety.

### BACKGROUND OF THE INVENTION

1. Field of the Invention

The present invention relates to a triarylamine derivative having absorption in a near infrared region.

2. Description of the Related Art

Conventionally, a diimmonium compound which is one kind of triarylamine derivative has been widely known as a near infrared absorbing agent (see, for example, Japanese Patent Application Laid-Open (JP-A) No. 4-146905; JP-A No. 5-142811; JP-A No. 2002-82219; “Der Deutchen Chemischen Gesellschaft” vol. 92, pp. 245-251 (1959); and “Spectrochemica Acta” vol. 23, pp. 655-675 (1967)), and has been widely used in a photopolymerization initiator, and an electrophotographic photoreceptor. Among these compounds, a diimmonium compound having an antimony hexafluoride ion as a counterion is very excellent in heat resistance. However, since the compound containing antimony is a deleterious substance, a diimmonium compound containing no antimony is desired in the industrial field. As a means to solve this problem, for example, a diimmonium compound having a perchlorate ion or a hexafluorophosphate ion as a counter ion is generally used.

### SUMMARY OF THE INVENTION

However, the diimmonium compound having a perchlorate ion or a hexafluorophosphate ion as a counter ion is insufficient in heat resistance and solubility.

An aspect of the present invention provides a triarylamine derivative represented by the following Formula (I).

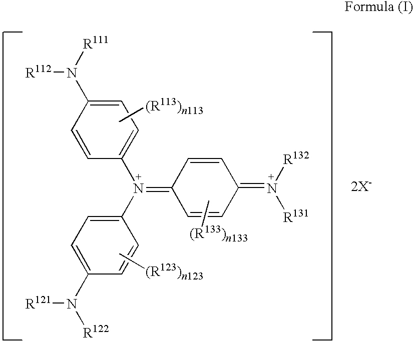

In Formula (I), R¹¹¹, R¹¹², R¹²¹, R¹²², R¹³¹ and R¹³² each independently represent a hydrogen atom, an aliphatic group or an aromatic group, provided that R¹³¹ and R¹³² are not both phenyl groups having a group represented by —NR¹⁴¹R¹⁴² at a fourth-position. R¹⁴¹ and R¹⁴² each independently represent a hydrogen atom, an aliphatic group or an aromatic group. R¹¹³, R¹²³ and R¹³³ each independently represent a substituent, and n₁₁₃, n₁₂₃ and n₁₃₃ each independently represent an integer of from 0 to 4. X⁻ represents a counteranion represented by any one of the following Formulae (II) to (IV).

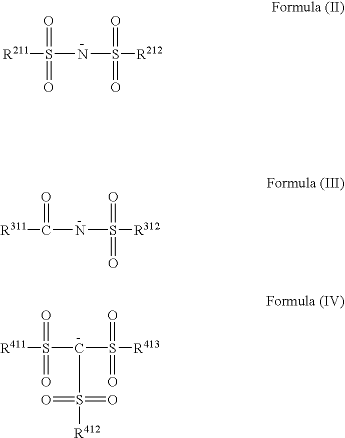

In Formulae (II) to (IV), R²¹¹, R²¹², R³¹¹, R³¹², R⁴¹¹, R⁴¹² and R⁴¹³ each independently represent an aliphatic group, an aromatic group or a heterocyclic group.

### DETAILED DESCRIPTION OF THE INVENTION

The triarylamine derivative according to the present invention is represented by the following Formula (I). Since the triarylamine derivative represented by the following Formula (I) or a tautomer thereof does not contain antimony, it is a near infrared absorbing diimmonium compound which has higher safety, and is excellent in heat resistance and solubility.

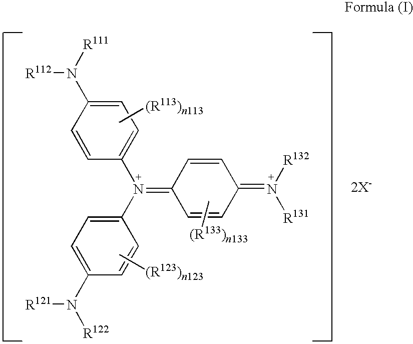

In Formula (I), R¹¹¹, R¹¹², R¹²¹, R¹²², R¹³¹ and R¹³² each independently represent a hydrogen atom, an aliphatic group or an aromatic group.

In the invention, the aliphatic group means an alkyl group, a substituted alkyl group, an alkenyl group, a substituted alkenyl group, an alkynyl group, a substituted alkynyl group, an aralkyl group or a substituted aralkyl group. The alkyl group may be branched, and may form a ring. The number of the carbon atoms in the alkyl group is preferably from 1 to 20, more preferably from 1 to 18. The scope of the alkyl moiety of a substituted alkyl group is the same as the above definition of an alkyl group. Specific examples of alkyl groups include a methyl group, an ethyl group, a n-propyl group, an isopropyl group, a t-butyl group, a n-octyl group, an eicosyl group, a 2-chloroethyl group, a 2-cyanoethyl group, a 2-ethylhexyl group, a cyclohexyl group, a cyclopentyl group, a 4-n-dodecyl cyclohexyl group, a bicyclo[1.2.2]heptan-2-yl group, and a bicyclo[2.2.2]octan-3-yl group.

The alkenyl group may be branched, and may form a ring. The number of the carbon atoms in the alkenyl group is preferably from 2 to 20, further preferably from 2 to 18. The scope of the alkenyl moiety of a substituted alkenyl group is the same as the above definition of an alkenyl group. Specific examples of alkenyl groups include a vinyl group, an allyl group, a prenyl group, a geranyl group, an oleyl group, a 2-cyclopenten-1-yl group, a 2-cyclohexen-1-yl group, a bicyclo[2.2.1]hept-2-en-1-yl group, and a bicyclo[2.2.2]oct-2-en-4-yl group.

The alkynyl group may be branched, and may form a ring. The number of the carbon atoms in the alkynyl group is preferably from 2 to 20, more preferably from 2 to 18. The scope of the alkynyl moiety of a substituted alkynyl group is the same as the above definition of an alkynyl group. Specific examples of alkynyl groups include an ethynyl group, a propargyl group, and a trimethylsilylethynyl group.

The scope of the alkyl moiety of an aralkyl group or a substituted aralkyl group is the same as the above definition of an alkyl group. The scope of the aryl moiety of an aralkyl group or a substituted aralkyl group is the same as the definition of an aryl group described below. Specific examples of aralkyl groups include a benzyl group and a phenylethyl group.

Examples of a substituent on the alkyl moiety of a substituted alkyl group, a substituted alkenyl group, a substituted alkynyl group or a substituted aralkyl group include the following substituents:

a halogen atom (e.g. a fluorine atom, a chlorine atom, a bromine atom, an iodine atom);

an alkyl group [a straight-chain, branched or cyclic, substituted or unsubstituted, alkyl group, wherein the scope of this alkyl group includes an alkyl group (preferably, an alkyl group having 1 to 30 carbon atoms, such as a methyl group, an ethyl group, a n-propyl group, an isopropyl group, a t-butyl group, a n-octyl group, an eicosyl group, a 2-chloroethyl group, a 2-cyanoethyl group, or a 2-ethylhexyl group), a cycloalkyl group (preferably, a substituted or unsubstituted cycloalkyl group having a 3 to 30 carbon atoms, such as a cyclohexyl group, a cyclopentyl group, or a 4-n-dodecylcyclohexyl group), a bicycloalkyl group (preferably, a substituted or unsubstituted bicycloalkyl group having 5 to 30 carbon atoms, which is a monovalent group obtained by removing one hydrogen atom from a bicycloalkane having 5 to 30 carbon atoms, such as a bicyclo[1.2.2]heptan-2-yl group or a bicyclo[2.2.2]octan-3-yl group), and an alkyl group having three or more cyclic structures, for example a tricyclo structure; alkyl groups in substituents described below (e.g. alkyl groups in alkylthio groups) are alkyl groups of the same concept];

an alkenyl group [a straight-chain, branched or cyclic, substituted or unsubstituted, alkenyl group, wherein the scope of this alkenyl group includes an alkenyl group (preferably, a substituted or unsubstituted alkenyl group having 2 to 30 carbon atoms, such as a vinyl group, an allyl group, a prenyl group, a geranyl group, or an oleyl group), a cycloalkenyl group (preferably a substituted or unsubstituted cycloalkenyl group having 3 to 30 carbon atoms, which is a monovalent group obtained by removing one hydrogen atom from a cycloalkene having 3 to 30 carbon atoms, such as a 2-cyclopenten-1-yl group or a 2-cyclohexen-1-yl group), and a bicycloalkenyl group (a substituted or unsubstituted bicycloalkenyl group, preferably a substituted or unsubstituted bicycloalkenyl group having 5 to 30 carbon atoms, which is a monovalent group obtained by removing one hydrogen atom from a bicycloalkene having one double bond, such as a bicyclo[2.2.1]hept-2-en-1-yl group or a bicyclo[2.2.2]oct-2-en-4-yl group)];

an alkynyl group (preferably, a substituted or unsubstituted alkynyl group having 2 to 30 carbon atoms, such as an ethynyl group, a propargyl group, or a trimethylsilylethynyl group);

an aryl group (preferably, a substituted or unsubstituted aryl group having 6 to 30 carbon atoms, such as a phenyl group, a p-tolyl group, a naphthyl group, a m-chlorophenyl group, or an o-hexadecanoylaminophenyl group);

a heterocyclic group (preferably a monovalent group obtained by removing one hydrogen atom from a 5- or 6-membered, substituted or unsubstituted, aromatic or non-aromatic heterocyclic compound, more preferably a 5- or 6-membered aromatic heterocyclic group having 3 to 30 carbon atoms, such as a 2-furyl group, a 2-thienyl group, a 2-pyrimidinyl group, or a 2-benzothiazolyl group);

a cyano group;

a hydroxy group;

a nitro group;

a carboxy group;

an alkoxy group (preferably, a substituted or unsubstituted alkoxy group having 1 to 30 carbon atoms, such as a methoxy group, an ethoxy group, an isopropyloxy group, a t-butoxy group, a n-octyloxy group, or a 2-methoxyethoxy group);

an aryloxy group (preferably, a substituted or unsubstituted aryloxy group having 6 to 30 carbon atoms, such as a phenoxy group, a 2-methylphenoxy group, a 4-t-butylphenoxy group, a 3-nitrophenoxy group, or a 2-tetradecanoylaminophenoxy group);

a silyloxy group (preferably, a silyloxy group having 3 to 20 carbon atoms, such as a trimethylsilyloxy group or a t-butyldimethylsilyloxy group);

a heterocyclic oxy group (preferably, a substituted or unsubstituted heterocyclic oxy group having 2 to 30 carbon atoms, a 1-phenyltetrazol-5-oxy group, or a 2-tetrahydropyranyloxy group);

an acyloxy group (preferably, a formyloxy group, a substituted or unsubstituted alkylcarbonyloxy group having 2 to 30 carbon atoms, or a substituted or unsubstituted arylcarbonyloxy group having 6 to 30 carbon atoms, such as a formyloxy group, an acetyloxy group, a pivaloyloxy group, a stearoyloxy group, a benzoyloxy group, or a p-methoxyphenylcarbonyloxy group);

a carbamoyloxy group (preferably, a substituted or unsubstituted carbamoyloxy group having 1 to 30 carbon atoms, such as a N,N-dimethylcarbamoyloxy group, a N,N-diethylcarbamoyloxy group, a morpholinocarbonyloxy group, a N,N-di-n-octylaminocarbonyloxy group, or a N-n-octylcarbamoyloxy group);

an alkoxycarbonyloxy group (preferably, a substituted or unsubstituted alkoxycarbonyloxy group having 2 to 30 carbon atoms, such as a methoxycarbonyloxy group, an ethoxycarbonyloxy group, a t-butoxycarbonyloxy group, or a n-octylcarbonyloxy group);

an aryloxycarbonyloxy group (preferably, a substituted or unsubstituted aryloxycarbonyloxy group having 7 to 30 carbon atoms, such as a phenoxycarbonyloxy group, a p-methoxyphenoxycarbonyloxy group, or a p-n-hexadecyloxyphenoxycarbonyloxy group);

an amino group (preferably, an amino group, a substituted or unsubstituted alkylamino group having 1 to 30 carbon atoms, or a substituted or unsubstituted anilino group having 6 to 30 carbon atoms, such as an amino group, a methylamino group, a dimethylamino group, an anilino group, a N-methyl-anilino group, or a diphenylamino group);

an acylamino group (preferably, a formylamino group, a substituted or unsubstituted alkylcarbonylamino group having 1 to 30 carbon atoms, or a substituted or unsubstituted arylcarbonylamino group having 6 to 30 carbon atoms, such as a formylamino group, an acetylamino group, a pivaloylamino group, a lauroylamino group, a benzoylamino group, or a 3,4,5-tri-n-octyloxyphenyl carbonylamino group);

an aminocarbonylamino group (preferably, a substituted or unsubstituted aminocarbonylamino group having 1 to 30 carbon atoms, such as a carbamoylamino group, a N,N-dimethylaminocarbonylamino group, a N,N-diethylaminocarbonylamino group, or a morpholinocarbonylamino group);

an alkoxycarbonylamino group (preferably, a substituted or unsubstituted alkoxycarbonylamino group having 2 to 30 carbon atoms, such as a methoxycarbonylamino group, an ethoxycarbonylamino group, a t-butoxycarbonylamino group, a n-octadecyloxycarbonylamino group, or a N-methyl-methoxycarbonylamino group);

an aryloxycarbonylamino group (preferably, a substituted or unsubstituted aryloxycarbonylamino group having 7 to 30 carbon atoms, such as a phenoxycarbonylamino group, a p-chlorophenoxycarbonylamino group, or a m-n-octyloxyphenoxycarbonylamino group);

a sulfamoylamino group (preferably, a substituted or unsubstituted sulfamoylamino group having 0 to 30 carbon atoms, such as a sulfamoylamino group, a N,N-dimethylaminosulfonylamino group, or a N-n-octylaminosulfonylamino group);

an alkyl- or aryl-sulfonylamino group (preferably, a substituted or unsubstituted alkylsulfonylamino group having 1 to 30 carbon atoms or a substituted or unsubstituted arylsulfonylamino group having 6 to 30 carbon atoms, such as a methylsulfonylamino group, a butylsulfonylamino group, a phenylsulfonylamino group, a 2,3,5-trichlorophenylsulfonylamino group, or a p-methylphenylsulfonylamino group);

a mercapto group;

an alkylthio group (preferably, a substituted or unsubstituted alkylthio group having 1 to 30 carbon atoms, such as a methylthio group, an ethylthio group, or a n-hexadecylthio group);

an arylthio group (preferably, a substituted or unsubstituted arylthio group having 6 to 30 carbon atoms, such as a phenylthio group, a p-chlorophenylthio group, or a m-methoxyphenylthio group);

a heterocyclic thio group (preferably, a substituted or unsubstituted heterocyclic thio group having 2 to 30 carbon atoms, such as a 2-benzothiazolylthio group or a 1-phenyltetrazol-5-ylthio group);

a sulfamoyl group (preferably, a substituted or unsubstituted sulfamoyl group having 0 to 30 carbon atoms, such as a N-ethylsulfamoyl group, a N-(3-dodecyloxypropyl)sulfamoyl group, a N,N-dimethylsulfamoyl group, a N-acetylsulfamoyl group, a N-benzoylsulfamoyl group, or a N-(N′-phenylcarbamoyl)sulfamoyl group);

a sulfo group;

an alkyl- or aryl-sulfinyl group (preferably, a substituted or unsubstituted alkylsulfinyl group having 1 to 30 carbon atoms or a substituted or unsubstituted arylsulfinyl group having 6 to 30 carbon atoms, such as a methylsulfinyl group, an ethylsulfinyl group, a phenylsulfinyl group, or a p-methylphenylsulfinyl group);

an alkyl- or aryl-sulfonyl group (preferably, a substituted or unsubstituted alkylsulfonyl group having 1 to 30 carbon atoms or a substituted or unsubstituted arylsulfonyl group having 6 to 30 carbon atoms, such as a methylsulfonyl group, an ethylsulfonyl group, a phenylsulfonyl group, a p-methylphenylsulfonyl group),

an acyl group (preferably, a formyl group, a substituted or unsubstituted alkylcarbonyl group having 2 to 30 carbon atoms, a substituted or unsubstituted arylcarbonyl group having 7 to 30 carbon atoms, or a substituted or unsubstituted heterocyclic carbonyl group having 4 to 30 carbon atoms and having a carbon atom bonded to the carbonyl group, such as an acetyl group, a pivaloyl group, a 2-chloroacetyl group, a stearoyl group, a benzoyl group, a p-n-octyloxyphenylcarbonyl group, a 2-pyridylcarbonyl group, or a 2-furylcarbonyl group);

an aryloxycarbonyl group (preferably, a substituted or unsubstituted aryloxycarbonyl group having 7 to 30 carbon atoms, such as a phenoxycarbonyl group, an o-chlorophenoxycarbonyl group, a m-nitrophenoxycarbonyl group, or a p-t-butylphenoxycarbonyl group);

an alkoxycarbonyl group (preferably, a substituted or unsubstituted alkoxycarbonyl group having 2 to 30 carbon atoms, such as a methoxycarbonyl group, an ethoxycarbonyl group, a t-butoxycarbonyl group, or a n-octadecyloxycarbonyl group);

a carbamoyl group (preferably, a substituted or unsubstituted carbamoyl group having 1 to 30 carbon atoms, such as a carbamoyl group, a N-methylcarbamoyl group, a N,N-dimethylcarbamoyl group, a N,N-di-n-octylcarbamoyl group, or a N-(methylsulfonyl)carbamoyl group);

aryl- and heterocyclyl-azo groups (preferably, a substituted or unsubstituted arylazo group having 6 to 30 carbon atoms or a substituted or unsubstituted heterocyclic azo group having 3 to 30 carbon atoms, such as a phenylazo group, a p-chlorophenylazo group, or a 5-ethylthio-1,3,4-thiadiazol-2-ylazo group);

an imido group (preferably, a N-succinimido group or a N-phthalimido group);

a phosphino group (preferably, a substituted or unsubstituted phosphino group having 2 to 30 carbon atoms, such as a dimethylphosphino group, a diphenylphosphino group, or a methylphenoxyphosphino group);

a phosphinyl group (preferably, a substituted or unsubstituted phosphinyl group having 2 to 30 carbon atoms, such as a phosphinyl group, a dioctyloxyphosphinyl group, or a diethoxyphosphinyl group);

a phosphinyloxy group (preferably, a substituted or unsubstituted phosphinyloxy group having 2 to 30 carbon atoms, such as a diphenoxyphosphinyloxy group or a dioctyloxyphosphinyloxy group);

a phosphinylamino group (preferably, a substituted or unsubstituted phosphinylamino group having 2 to 30 carbon atoms, such as a dimethoxyphosphinylamino group or a dimethylaminophosphinylamino group); and

a silyl group (preferably, a substituted or unsubstituted silyl group having 3 to 30 carbon atoms, such as a trimethylsilyl group, a t-butyldimethylsilyl group, or a phenyldimethylsilyl group).

The scope of substituents also include those obtained by replacing hydrogen atoms in substituents having hydrogen atoms, among the above-mentioned substituents, with any of the above-mentioned substituents. Examples thereof include an alkylcarbonylaminosulfonyl group, an arylcarbonylaminosulfonyl group, an alkylsulfonylaminocarbonyl group, and an arylsulfonylaminocarbonyl group. More specific examples include a methylsulfonylaminocarbonyl group, a p-methylphenylsulfonylaminocarbonyl group, an acetylaminosulfonyl group, and a benzoylaminosulfonyl group.

When the aliphatic group is a substituted aralkyl group, examples of a substituent on the aryl moiety of the substituted aralkyl group are the same as examples of a substituent of a substituted aryl group described below.

In the invention, an aromatic group means an aryl group or a substituted aryl group. The aromatic group may be fused with an aliphatic ring, another aromatic ring or a heterocyclic ring. The number of the carbon atoms in the aryl group is preferably from 6 to 40, more preferably from 6 to 30, further preferably from 6 to 20. In particular, the aryl group is preferably a phenyl group or a naphthyl group, particularly preferably a phenyl group.

The above description on the aryl group applies also to the aryl moiety of a substituted aryl group. Examples of substituents of substituted aryl groups include the same substituents as those listed above as examples of substituents on the alkyl moieties of a substituted alkyl group, a substituted alkenyl group, a substituted alkynyl group and a substituted aralkyl group.

In the invention, each of R¹¹¹, R¹¹², R¹²¹, R¹²², R¹³¹ and R¹³² is preferably a hydrogen atom, a substituted or unsubstituted alkyl group, a substituted or unsubstituted alkenyl group, a substituted or unsubstituted alkynyl group, or a substituted or unsubstituted aryl group, more preferably a hydrogen atom, a substituted or unsubstituted alkyl group having a total carbon number of from 1 to 20, a substituted or unsubstituted alkenyl group having a total carbon number of from 2 to 20, a substituted or unsubstituted alkynyl group having a total carbon number of from 2 to 20, or a substituted or unsubstituted aryl group having a total carbon number of from 6 to 20, still more preferably a substituted or unsubstituted alkyl group having a total carbon number of from 1 to 10, a substituted or unsubstituted alkenyl group having a total carbon number of from 2 to 10, or a substituted or unsubstituted aryl group having a total carbon number of from 6 to 10, further preferably a substituted or unsubstituted alkyl group having a total carbon number of from 1 to 8, or a substituted or unsubstituted aryl group having a total carbon number of from 6 to 8, most preferably a substituted or unsubstituted alkyl group having a total carbon number of from 2 to 6. It is also preferable that all of R¹¹¹, R¹¹², R¹²¹, R¹²², R¹³¹ and R¹³² are the same.

In the invention, from a viewpoint of light resistance, heat resistance, and wet heat resistance, R¹³¹ and R¹³² are not phenyl groups each having a group represented by —NR¹⁴¹R¹⁴² at a 4-position at the same time. Herein, R¹⁴¹ and R¹⁴² each independently represent a hydrogen atom, an aliphatic group or an aromatic group.

In the invention, all of R¹¹¹, R¹¹², R¹²¹, R¹²², R¹³¹ and R¹³² are preferably branched alkyl groups, more preferably branched alkyl groups each having a carbon number of from 3 to 10, more preferably branched alkyl groups each having a carbon number of from 3 to 8, particularly preferably branched alkyl groups each having a carbon number of from 3 to 6.

In the invention, at least one of R¹¹¹, R¹¹², R¹²¹, R¹²², R¹³¹ and R₁₃₂ is preferably an alkyl group having a cyano group, more preferably, an alkyl group having a cyano group and a total carbon number of from 2 to 11, still more preferably an alkyl group having a cyano group and a total carbon number of from 2 to 9, particularly preferably an alkyl group having a cyano group and a total carbon number of 3 to 7.

Further, in the invention, at least one of R¹¹¹, R¹¹², R¹²¹, R¹²², R¹³¹ and R¹³² is preferably an alkyl group having a halogen atom, more preferably an alkyl group having a halogen atom and a total carbon number of 1 to 10, still more preferably an alkyl group having a halogen atom and a total carbon number of 1 to 8, particularly preferably an alkyl group having a halogen atom and a total carbon number of 2 to 6.

From a viewpoint of light resistance, the halogen atom is preferably a fluorine atom.

In Formula (I), R¹¹³, R¹²³ and R¹³³ each independently represent a substituent, and examples of the substituent include the same substituents as those listed as examples of substituents on the alkyl moieties of a substituted alkyl group, a substituted alkenyl group, a substituted alkynyl group and a substituted aralkyl group.

In the invention, R¹¹³, R¹²³ and R¹³³ each independently represent, preferably a halogen atom, an alkyl group, an alkenyl group, an alkynyl group, an aryl group, a cyano group, a hydroxyl group, a carboxy group, an alkoxy group, an aryloxy group, a silyloxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl and arylsulfonyl amino group, a mercapto group, an alkylthio group, an arylthio group, a sulfamoyl group, a sulfo group, an alkyl- or aryl-sulfinyl group, an alkyl- or aryl-sulfonyl group, an acyl group, an aryloxycarbonyl group, an alkoxycarbonyl group, carbamoyl group, an imido group, a phosphino group, a phosphinyl group, a phosphinyloxy group, a phosphinylamino group or a silyl group, more preferably, a halogen atom, an alkyl group, an alkynyl group, an aryl group, a cyano group, a hydroxy group, a carboxy group, an alkoxy group, an aryloxy group, a silyloxy group, an amino group, an alkylthio group, an arylthio group, an imido group, or a silyl group, still more preferably a halogen atom, an alkyl group, an aryl group, an alkoxy group, an aryloxy group, a silyloxy group or an amino group, most preferably an alkyl group or an alkoxy group. It is also preferably that all of R¹¹³, R¹²³ and R¹³³ are the same.

Regarding substitution positions of R¹¹³, R¹²³ and R¹³³, any of a 2-position and a 3-position may be selected.

In Formula (I), n₁₁₃, n₁₂₃ and n₁₃₃ each independently represent an integer of 0 to 4, preferably an integer of 0 to 3, more preferably an integer of 0 to 2, still more preferably 0 or 1, most preferably 0.

In Formula (I), X⁻ represents a counteranion represented by any one of the following Formula (II) to Formula (IV), or a tautomer thereof. Thereby, an infrared absorbing triarylamine derivative which has higher safety, and has good heat resistance and solubility may be obtained.

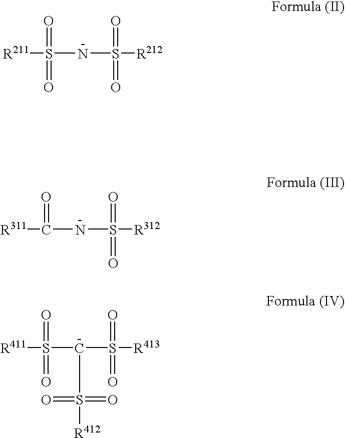

In Formulae, R²¹¹, R²¹², R³¹¹, R³¹², R⁴¹¹, R⁴¹² and R⁴¹³ each independently represent an aliphatic group, an aromatic group or a heterocyclic group. The aliphatic group and the aromatic group are as described above. Examples of the heterocyclic group include heterocyclic groups listed above as examples of substituents on the alkyl moieties of a substituted alkyl group, a substituted alkenyl group, a substituted alkynyl group and a substituted aralkyl group.

In the invention, R²¹¹, R²¹², R³¹¹, R³¹², R⁴¹¹, R⁴¹² and R⁴¹³ each independently represent, preferably an alkyl group, an alkenyl group, an alkynyl group, an aryl group, or a heterocyclic group, more preferably an alkyl group having a carbon number of from 1 to 20, an alkenyl group having a carbon number of from 2 to 20, an alkynyl group having a carbon number of from 2 to 20 or an aryl group having a carbon number of from 6 to 20, still more preferably an alkyl group having a carbon number of from 1 to 10, an alkenyl group having a carbon number of from 2 to 10 or an aryl group having a carbon number of from 6 to 10, further preferably an alkyl group having a carbon number of from 1 to 8 or an aryl group having a carbon number of from 6 to 8, most preferably an alkyl group having a carbon number of from 1 to 6. Among them, a perfluoroalkyl group represented by CₙF₂ₙ₊₁ is desirable, wherein n represents an integer of from 1 to 6.

In the compound represented by Formula (I) in the invention, from viewpoints of heat resistance and solubility, it is preferable that R¹¹¹, R¹¹², R¹²¹, R¹²², R¹³¹ and R¹³² each independently represent a hydrogen atom, an alkyl group, or an aryl group, n₁₁₃, n₁₂₃ and n₁₃₃ each independently represent 0 or 1, R¹¹³, R¹²³ and R¹³³ each independently represent an alkyl group or an alkoxy group, and R²¹¹, R²¹², R³¹¹, R³¹², R⁴¹¹, R⁴¹² and R⁴¹³ each independently represent an alkyl group or an aryl group, and it is more preferable that R¹¹¹, R¹¹², R¹²¹, R¹²², R¹³¹ and R¹³² each independently represent an alkyl group having a carbon number of from 2 to 6, n₁₁₃, n₁₂₃ and n₁₃₃ represent 0, and R²¹¹, R²¹², R³¹¹, R³¹², R⁴¹¹, R⁴¹² and R⁴¹³ each independently represent a perfluoroalkyl group having a carbon number of from 1 to 6.

In the invention, it is also preferable that all of R¹¹¹, R¹¹², R¹²¹, R¹²², R¹³¹ and R¹³² each independently represent a branched alkyl group having a carbon number of 2 to 6, n₁₁₃, n₁₂₃ and n₁₃₃ represent 0, and R²¹¹, R²¹², R³¹¹, R³¹², R⁴¹¹, R⁴¹² and R⁴¹³ each independently represent a perfluoroalkyl group having a carbon number of 1 to 6.

In addition, it is also preferable that at least one of R¹¹¹, R¹¹², R¹²¹, R¹²², R¹³¹ and R¹³² is an alkyl group having a cyano group and a carbon number of 2 to 6, n₁₁₃, n₁₂₃ and n₁₃₃ represent 0, and R²¹¹, R²¹², R³¹¹, R³¹², R⁴¹¹, R⁴¹² and R⁴¹³ each independently represent a perfluoroalkyl group having a carbon number of 1 to 6.

In addition, it is also preferable that at least one of R¹¹¹, R¹¹², R¹²¹, R¹²², R¹³¹ and R¹³² is an alkyl group having a halogen atom and a carbon number of 2 to 6, n₁₁₃, n₁₂₃ and n₁₃₃ represent 0, and R²¹¹, R²¹², R³¹¹, R³¹², R⁴¹¹, R⁴¹² and R⁴¹³ each independently represent a perfluoroalkyl group having a carbon number of 1 to 6, and it is more preferable that the halogen atom is a fluorine atom.

As specific examples of triarylamine derivatives represented by Formula (I) according to the invention, compounds 1 to 142 are illustrated below, but the invention is not limited to them.

In the compound represented by Formula (I), specific examples of a diimmonium compound in which X⁻ is represented by Formula (II) (compounds 1 to 40, and 131 to 134) are illustrated in Table 1 below and separate chemical formulae, specific examples of a diimmonium compound in which X⁻ is represented by Formula (III) (compounds 41 to 80, and 135 to 138) are illustrated in Table 2 below and separate chemical formulae, and specific examples of a diimmonium compound in which X⁻ is represented by Formula (IV) (compounds 81 to 130, and 139 to 142) are illustrated in Table 3 and Table 4 below and separate chemical formulae.

TABLE 1

| No. | (R¹¹¹, R¹¹²), (R¹²¹, R¹²²), (R¹³¹, R¹³²) | R¹¹³, R¹²³, R¹³³ | (R²¹¹, R²¹²) |
|---|---|---|---|
| 1 | 3 (CH₃, CH₃) | — | (CF₃, CF₃) |
| 2 | 3 (C₂H₅, C₂H₅) | — | (CF₃, CF₃) |
| 3 | 3 (n-C₃H₇, n-C₃H₇) | — | (CF₃, CF₃) |
| 4 | 3 (iso-C₃H₇, iso-C₃H₇) | — | (CF₃, CF₃) |
| 5 | 3 (n-C₄H₉, n-C₄H₉) | — | (CF₃, CF₃) |
| 6 | 3 (iso-C₄H₉, iso-C₄H₉) | — | (CF₃, CF₃) |
| 7 | 3 (sec-C₄H₉, sec-C₄H₉) | — | (CF₃, CF₃) |
| 8 | 3 (tert-C₄H₉, tert-C₄H₉) | — | (CF₃, CF₃) |
| 9 | 3 (n-C₅H₁₁, n-C₅H₁₁) | — | (CF₃, CF₃) |
| 10 | 3 (iso-C₅H₁₁, iso-C₅H₁₁) | — | (CF₃, CF₃) |
| 11 | 3 (n-C₆H₁₃, n-C₆H₁₃) | — | (CF₃, CF₃) |
| 12 | 4 (cyclo-C₆H₁₁, cyclo-C₆H₁₁) | — | (CF₃, CF₃) |
| 13 | 3 (C₂H₄CN, C₂H₄CN) | — | (CF₃, CF₃) |
| 14 | 3 (C₃H₆CN, C₃H₆CN) | — | (CF₃, CF₃) |
| 15 | 3 (CF₃, CF₃) | — | (CF₃, CF₃) |
| 16 | 3 (C₂F₅, C₂F₅) | — | (CF₃, CF₃) |
| 17 | 3 (CF₂CF₂CF₃, CF₂CF₂CF₃) | — | (CF₃, CF₃) |
| 18 | 3 (CH₂CH₂CF₃, CH₂CH₂CF₃) | — | (CF₃, CF₃) |
| 19 | 3 (CH₂CH₂OCH₃, CH₂CH₂OCH₃) | — | (CF₃, CF₃) |
| 20 | 3 (CH₂CH₂COOH, CH₂CH₂COOH) | — | (CF₃, CF₃) |
| 21 | 3 (H, H) | — | (CF₃, CF₃) |
| 22 | 3 (Ph, Ph) | — | (CF₃, CF₃) |
| 23 | 3 (n-C₃H₇, n-C₃H₇) | — | (CF₃, C₂F₅) |
| 24 | 3 (iso-C₃H₇, iso-C₃H₇) | — | (CF₃, C₂F₅) |
| 25 | 3 (n-C₄H₉, n-C₄H₉) | — | (CF₃, C₂F₅) |
| 26 | 3 (iso-C₄H₉, iso-C₄H₉) | — | (CF₃, C₂F₅) |
| 27 | 3 (C₃H₆CN, C₃H₆CN) | — | (CF₃, C₂F₅) |
| 28 | 3 (C₂F₅, C₂F₅) | — | (CF₃, C₂F₅) |
| 29 | 3 (CF₂CF₂CF₃, CF₂CF₂CF₃) | — | (CF₃, C₂F₅) |
| 30 | 3 (CH₂CH₂CF₃, CH₂CH₂CF₃) | — | (CF₃, C₂F₅) |
| 31 | 3 (H, H) | — | (CF₃, C₂F₅) |
| 32 | 3 (Ph, Ph) | — | (CF₃, C₂F₅) |
| 33 | 3 (n-C₄H₉, n-C₄H₉) | — | (CF₃, C₃F₇) |
| 34 | 3 (n-C₄H₉, n-C₄H₉) | — | (C₂F₅, C₃F₇) |
| 35 | 3 (n-C₄H₉, n-C₄H₉) | — | (CF₃, CF₃) |
| 36 | 3 (n-C₄H₉, n-C₄H₉) | — | (CF₃, C₆H₄CF₃) |
| 37 | 3 (n-C₄H₉, n-C₄H₉) | — | (CF₃, C₆H₄CF₃) |
| 38 | 3 (n-C₄H₉, n-C₄H₉) | 3 CH₃ | (CF₃, CF₃) |
| 39 | 3 (n-C₄H₉, n-C₄H₉) | 3 C₂H₅ | (CF₃, CF₃) |
| 40 | 3 (n-C₄H₉, n-C₄H₉) | 3 (OCH₃) | (CF₃, CF₃) |

TABLE 2

| No. | (R¹¹¹, R¹¹²), (R¹²¹, R¹²²), (R¹³¹, R¹³²) | R¹¹³, R¹²³, R¹³³ | (R³¹¹, R³¹²) |
|---|---|---|---|
| 41 | 3 (CH₃, CH₃) | — | (CF₃, CF₃) |
| 42 | 3 (C₂H₅, C₂H₅) | — | (CF₃, CF₃) |
| 43 | 3 (n-C₃H₇, n-C₃H₇) | — | (CF₃, CF₃) |
| 44 | 3 (iso-C₃H₇, iso-C₃H₇) | — | (CF₃, CF₃) |
| 45 | 3 (n-C₄H₉, n-C₄H₉) | — | (CF₃, CF₃) |
| 46 | 3 (iso-C₄H₉, iso-C₄H₉) | — | (CF₃, CF₃) |
| 47 | 3 (sec-C₄H_{9,} sec-C₄H₉) | — | (CF₃, CF₃) |
| 48 | 3 (tert-C₄H₉, tert-C₄H₉) | — | (CF₃, CF₃) |
| 49 | 3 (n-C₅H₁₁, n-C₅H₁₁) | — | (CF₃, CF₃) |
| 50 | 3 (iso-C₅H₁₁, iso-C₅H₁₁) | — | (CF₃, CF₃) |
| 51 | 3 (n-C₆H₁₃, n-C₆H₁₃) | — | (CF₃, CF₃) |
| 52 | 4 (cyclo-C₆H₁₁, cyclo-C₆H₁₁) | — | (CF₃, CF₃) |
| 53 | 3 (C₂H₄CN, C₂H₄CN) | — | (CF₃, CF₃) |
| 54 | 3 (C₃H₆CN, C₃H₆CN) | — | (CF₃, CF₃) |
| 55 | 3 (CF₃, CF₃) | — | (CF₃, CF₃) |
| 56 | 3 (C₂F₅, C₂F₅) | — | (CF₃, CF₃) |
| 57 | 3 (CF₂CF₂CF₃, CF₂CF₂CF₃) | — | (CF₃, CF₃) |
| 58 | 3 (CH₂CH₂CF₃, CH₂CH₂CF₃) | — | (CF₃, CF₃) |
| 59 | 3 (CH₂CH₂OCH₃, CH₂CH₂OCH₃) | — | (CF₃, CF₃) |
| 60 | 3 (CH₂CH₂COOH, CH₂CH₂COOH) | — | (CF₃, CF₃) |
| 61 | 3 (H, H) | — | (CF₃, CF₃) |
| 62 | 3 (Ph, Ph) | — | (CF₃, CF₃) |
| 63 | 3 (n-C₃H₇, n-C₃H₇) | — | (CF₃, C₂F₅) |
| 64 | 3 (iso-C₃H₇, iso-C₃H₇) | — | (CF₃, C₂F₅) |
| 65 | 3 (n-C₄H₉, n-C₄H₉) | — | (CF₃, C₂F₅) |
| 66 | 3 (iso-C₄H₉, iso-C₄H₉) | — | (CF₃, C₂F₅) |
| 67 | 3 (C₃H₆CN, C₃H₆CN) | — | (CF₃, C₂F₅) |
| 68 | 3 (C₂F₅, C₂F₅) | — | (CF₃, C₂F₅) |
| 69 | 3 (CF₂CF₂CF₃, CF₂CF₂CF₃) | — | (CF₃, C₂F₅) |
| 70 | 3 (CH₂CH₂CF₃, CH₂CH₂CF₃) | — | (CF₃, C₂F₅) |
| 71 | 3 (H, H) | — | (CF₃, C₂F₅) |
| 72 | 3 (Ph, Ph) | — | (CF₃, C₂F₅) |
| 73 | 3 (n-C₄H₉, n-C₄H₉) | — | (CF₃, C₃F₇) |
| 74 | 3 (n-C₄H₉, n-C₄H₉) | — | (C₂F_{5,} C₃F₇) |
| 75 | 3 (n-C₄H₉, n-C₄H₉) | — | (CF₃, CF₃) |
| 76 | 3 (n-C₄H₉, n-C₄H₉) | — | (CF₃, C₆H₄CF₃) |
| 77 | 3 (n-C₄H₉, n-C₄H₉) | — | (CF₃, C₆H₄CF₃) |
| 78 | 3 (n-C₄H₉, n-C₄H₉) | 3 CH₃ | (CF₃, CF₃) |
| 79 | 3 (n-C₄H₉, n-C₄H₉) | 3 C₂H₅ | (CF₃, CF₃) |
| 80 | 3 (n-C₄H₉, n-C₄H₉) | 3 (OCH₃) | (CF₃, CF₃) |

TABLE 3

| No. | (R¹¹¹, R¹¹²), (R¹²¹, R¹²²), (R¹³¹, R¹³²) | R¹¹³, R¹²³, R¹³³ | (R⁴¹¹, R⁴¹², R⁴¹³) |
|---|---|---|---|
| 81 | 3 (CH₃, CH₃) | — | (CF₃, CF₃, CF₃) |
| 82 | 3 (C₂H₅, C₂H₅) | — | (CF₃, CF₃, CF₃) |
| 83 | 3 (n-C₃H₇, n-C₃H₇) | — | (CF₃, CF₃, CF₃) |
| 84 | 3 (iso-C₃H₇, iso-C₃H₇) | — | (CF₃, CF₃, CF₃) |
| 85 | 3 (n-C₄H₉, n-C₄H₉) | — | (CF₃, CF₃, CF₃) |
| 86 | 3 (iso-C₄H₉, iso-C₄H₉) | — | (CF₃, CF₃, CF₃) |
| 87 | 3 (sec-C₄H₉, sec-C₄H₉) | — | (CF₃, CF₃, CF₃) |
| 88 | 3 (tert-C₄H₉, tert-C₄H₉) | — | (CF₃, CF₃, CF₃) |
| 89 | 3 (n-C₅H₁₁, n-C₅H₁₁) | — | (CF₃, CF₃, CF₃) |
| 90 | 3 (iso-C₅H₁₁, iso-C₅H₁₁) | — | (CF₃, CF₃, CF₃) |
| 91 | 3 (n-C₆H₁₃, n-C₆H₁₃) | — | (CF₃, CF₃, CF₃) |
| 92 | 4 (cyclo-C₆H₁₁, cyclo-C₆H₁₁) | — | (CF₃, CF₃, CF₃) |
| 93 | 3 (C₂H₄CN, C₂H₄CN) | — | (CF₃, CF₃, CF₃) |
| 94 | 3 (C₃H₆CN, C₃H₆CN) | — | (CF₃, CF₃, CF₃) |
| 95 | 3 (CF_{3,} CF₃) | — | (CF₃, CF₃, CF₃) |
| 96 | 3 (C₂F_{5,} C₂F₅) | — | (CF₃, CF₃, CF₃) |
| 97 | 3 (CF₂CF₂CF₃, CF₂CF₂CF₃) | — | (CF₃, CF₃, CF₃) |
| 98 | 3 (CH₂CH₂CF₃, CH₂CH₂CF₃) | — | (CF₃, CF₃, CF₃) |
| 99 | 3 (CH₂CH₂OCH₃, CH₂CH₂OCH₃) | — | (CF₃, CF₃, CF₃) |
| 100 | 3 (CH₂CH₂COOH, CH₂CH₂COOH) | — | (CF₃, CF₃, CF₃) |
| 101 | 3 (H, H) | — | (CF₃, CF₃, CF₃) |
| 102 | 3 (Ph, Ph) | — | (CF₃, CF₃, CF₃) |
| 103 | 3 (n-C₃H₇, n-C₃H₇) | — | (CF₃, CF₃, C₂F₅) |
| 104 | 3 (iso-C₃H₇, iso-C₃H₇) | — | (CF₃, CF₃, C₂F₅) |
| 105 | 3 (n-C₄H₉, n-C₄H₉) | — | (CF₃, CF₃, C₂F₅) |
| 106 | 3 (iso-C₄H₉, iso-C₄H₉) | — | (CF₃, CF₃, C₂F₅) |
| 107 | 3 (C₃H₆CN, C₃H₆CN) | — | (CF₃, CF₃, C₂F₅) |
| 108 | 3 (C₂F₅, C₂F₅) | — | (CF₃, CF₃, C₂F₅) |
| 109 | 3 (CF₂CF₂CF₃, CF₂CF₂CF₃) | — | (CF₃, CF₃, C₂F₅) |
| 110 | 3 (CH₂CH₂CF₃, CH₂CH₂CF₃) | — | (CF₃, CF₃, C₂F₅) |
| 111 | 3 (H, H) | — | (CF₃, CF₃, C₂F₅) |
| 112 | 3 (Ph, Ph) | — | (CF₃, CF₃, C₂F₅) |

TABLE 4

| No. | (R¹¹¹, R¹¹²), (R¹²¹, R¹²²), (R¹³¹, R¹³²) | R¹¹³, R¹²³, R¹³³ | (R⁴¹¹, R⁴¹², R⁴¹³) |
|---|---|---|---|
| 113 | 3 (n-C₄H₉, n-C₄H₉) | — | (CF₃, C₂F₅, C₂F₅) |
| 114 | 3 (n-C₄H₉, n-C₄H₉) | — | (C₂F₅, C₂F₅, C₂F₅) |
| 115 | 3 (n-C₄H₉, n-C₄H₉) | — | (C₂F₅, C₂F₅, C₃F₇) |
| 116 | 3 (n-C₄H₉, n-C₄H₉) | — | (CF_{3,} C₂F₅, C₃F₇) |
| 117 | 3 (n-C₄H₉, n-C₄H₉) | — | (CF₃, CF₃, CH₃) |
| 118 | 3 (n-C₄H₉, n-C₄H₉) | — | (CF₃, CH₃, CH₃) |
| 119 | 3 (n-C₄H₉, n-C₄H₉) | — | (CF₃, CF₃, iso-C₅H₁₁) |
| 120 | 3 (n-C₄H₉, n-C₄H₉) | — | (C₂F₅, C₂F₅, iso-C₅H₁₁) |
| 121 | 3 (n-C₄H₉, n-C₄H₉) | — | (C₂F₅, CH₃, C₆H₄CH₃) |
| 122 | 3 (n-C₄H₉, n-C₄H₉) | 3 CH₃ | (CF₃, CF₃, CF₃) |
| 123 | 3 (n-C₄H₉, n-C₄H₉) | 3 C₂H₅ | (CF₃, CF₃, CF₃) |
| 124 | 3 (n-C₄H₉, n-C₄H₉) | 3 (OCH₃) | (CF₃, CF₃, CF₃) |
| 125 | 3 (n-C₄H₉, n-C₄H₉) | 3 CH₃ | (CF₃, C₂F_{5,} C₂F₅,) |
| 126 | 3 (n-C₄H₉, n-C₄H₉) | 3 C₂H₅ | (CF₃, C₂F_{5,} C₂F₅,) |
| 127 | 3 (n-C₄H₉, n-C₄H₉) | 3 (OCH₃) | (CF₃, C₂F_{5,} C₂F₅,) |
| 128 | 3 (n-C₄H₉, n-C₄H₉) | 3 CH₃ | (C₂F₅, CH₃, C₆H₄CH₃) |
| 129 | 3 (n-C₄H₉, n-C₄H₉) | 3 C₂H₅ | (C₂F₅, CH₃, C₆H₄CH₃) |
| 130 | 3 (n-C₄H₉, n-C₄H₉) | 3 (OCH₃) | (C₂F₅, CH₃, C₆H₄CH₃) |

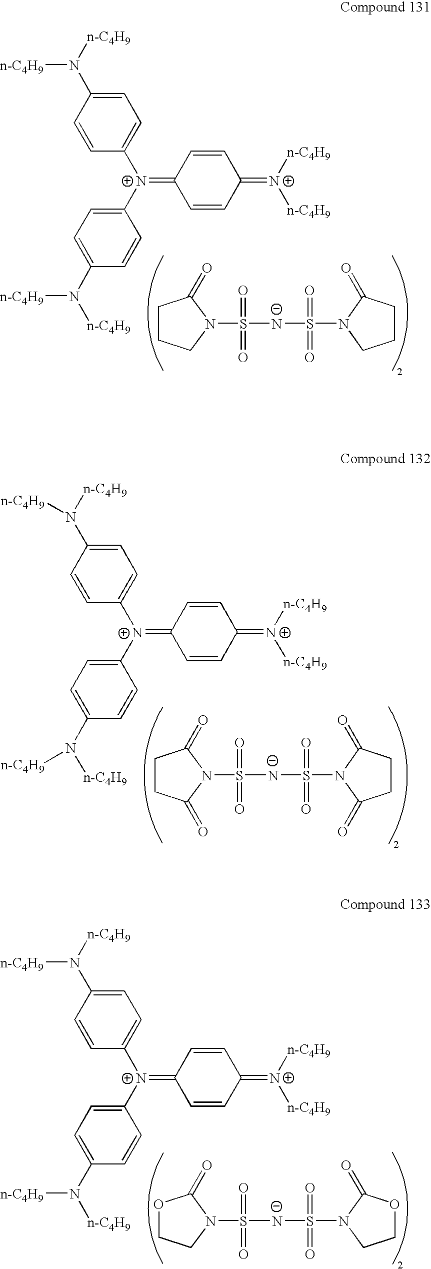

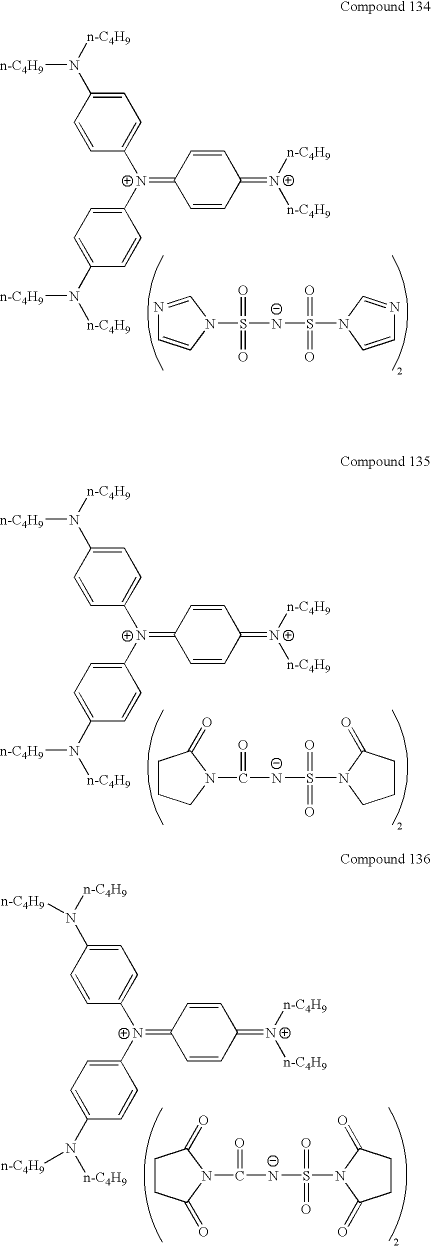

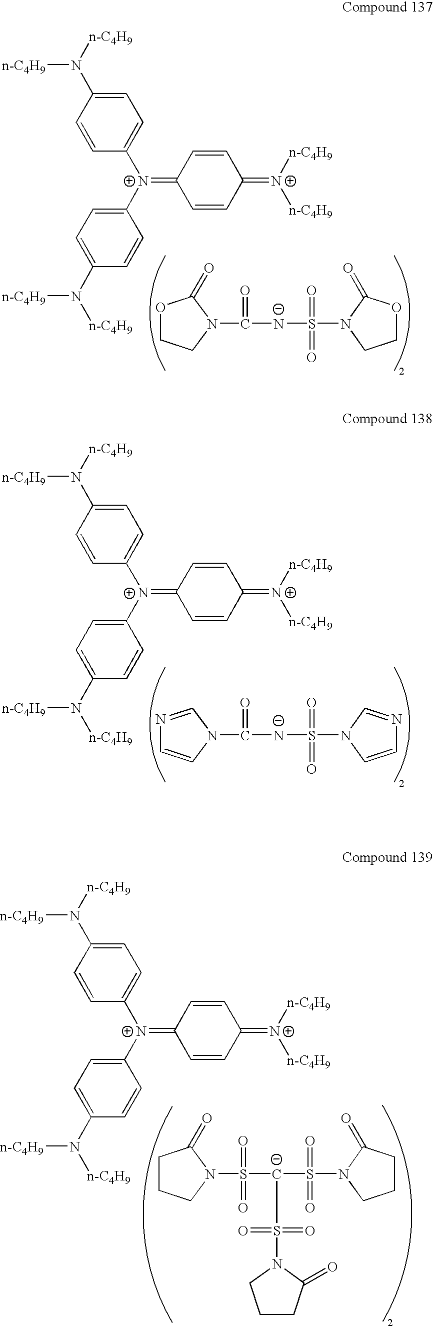

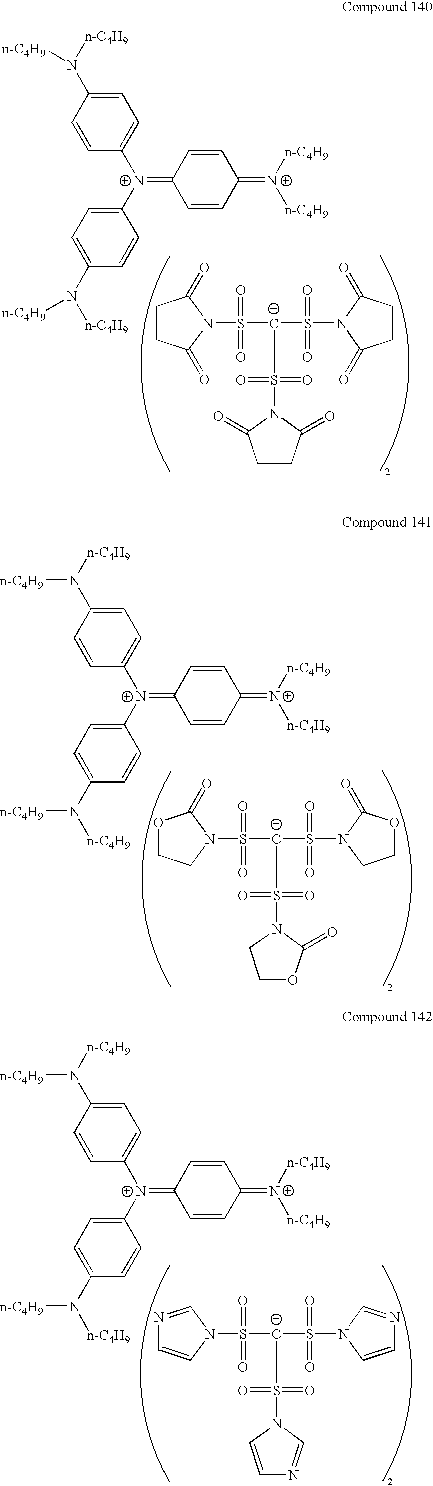

In the invention, the compound represented by Formula (I) may be synthesized, for example, by treating a compound represented by Formula (V) with a metal salt (preferably, a silver salt) of an anion represented by any one of Formulae (II) to (IV).

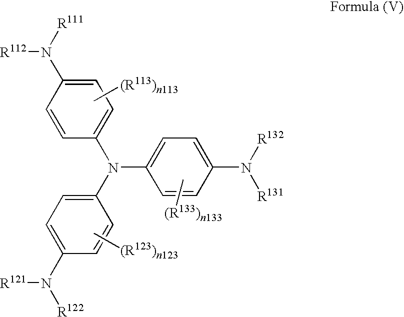

In Formula (V), R¹¹¹, R¹¹², R¹²¹, R¹²², R¹³¹, R¹³², R¹¹³, R¹²³, R¹³³, n₁₁₃, n₁₂₃ and n₁₃₃ have the same definitions as those of R¹¹¹, R¹¹², R¹²¹, R¹²², R¹³¹, R¹³², R¹¹³, R¹²³, R¹²³, R¹¹³, n₁₁₃, n₁₂₃ and n₁₃₃ in Formula (I), respectively.

The compound represented by Formula (V) may be synthesized, for example, based on the method described in “Der Deutschen Chemischen Gesellschaft” vol. 92, pp. 245-251 (1959). Silver salts of anions represented by Formula (II) to Formula (IV) may be synthesized, for example, based on the method described in JP-A No. 2005-325292, and WO 04-048480.

In synthesis of the compound represented by Formula (I), as a ratio of raw materials used in the synthesis reaction, a silver salt of an anion represented by any one of Formula (II) to Formula (IV) is used at preferably 0.1 to 10 moles, more preferably 1 to 6 moles, further preferably 1.5 to 5 moles, particularly preferably 2 to 4 moles relative to 1 mole of the compound represented by Formula (V), whereby a compound represented by Formula (I) may be obtained.

Examples of a solvent used in the synthesis reaction include water, an amide solvent (e.g. N,N-dimethylformamide, N,N-dimethylacetamide, 1-methyl-2-pyrrolidone), a sulfone solvent (e.g. sulfolane), a sulfoxide solvent (e.g. dimethyl sulfoxide), a ureide solvent (e.g. tetramethylurea), an ether solvent (e.g. dioxane, cyclopentyl methyl ether), a ketone solvent (e.g. acetone, cyclohexanone), a hydrocarbon solvent (e.g. toluene, xylene, n-decane), a halogen solvent (e.g. tetrachloroethane, chlorobenzene), an alcohol solvent (e.g. methanol, ethanol, isopropyl alcohol, ethylene glycol, cyclohexanol, phenol), a pyridine solvent (e.g. pyridine, γ-picoline, 2,6-lutidine), an ester solvent (e.g. ethyl acetate, butyl acetate), a carboxylic acid solvent (e.g. acetic acid, propionic acid), and a nitrile solvent (e.g. acetonitrile). In an embodiment, only one solvent, which may be selected from the above, is used singly. In another embodiment, two or more solvents, which may be selected from the above, are used together.

Among these solvents, preferable examples include water, an amide solvent, a sulfone solvent, a sulfoxide solvent, a ureide solvent, a halogen solvent, an alcohol solvent, a pyridine solvent, an ester solvent, a carboxylic acid solvent and a nitrile solvent, more preferable examples include water, an amide solvent, a sulfone solvent, a ureide solvent, a halogen solvent, an alcohol solvent, an ester solvent and a nitrile solvent, and still more preferable examples include water, a sulfone solvent, an alcohol solvent, an ester solvent and a nitrile solvent. It is also preferable that water and one or more other solvents are used together.

The reaction temperature may be from −30 to 250° C., preferably from −0.10 to 150° C., more preferably from −5 to 100° C., still more preferably from 0 to 70° C., further preferably from 10 to 50° C. It is also preferable to carry out the reaction initially at −5 to 20° C. and then raise the reaction temperature to a range of from 25 to 100° C.

Since a triarylamine derivative represented by Formula (I) according to the invention is a near infrared absorbing diimmonium compound which has good heat resistance, it may be widely used, for example, in photopolymerization initiators and electrophotographic photoreceptors.

In the following, exemplary embodiments are described:

<1>. A triarylamine derivative represented by the following Formula (I):

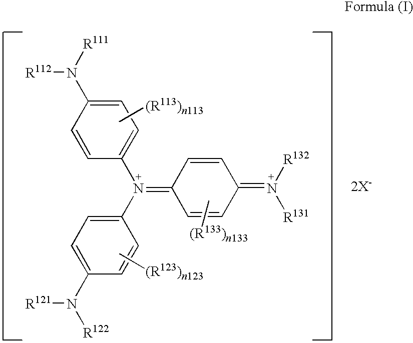

wherein, in Formula (I): R¹¹¹, R¹¹², R¹²¹, R¹²², R¹³¹ and R¹³² each independently represent a hydrogen atom, an aliphatic group or an aromatic group, provided that R¹³¹ and R¹³² are not both phenyl groups having a group represented by —NR¹⁴¹R¹⁴² at a fourth position; R¹⁴¹ and R¹⁴² each independently represent a hydrogen atom, an aliphatic group, or an aromatic group; R¹¹³, R¹²³ and R¹³³ each independently represent a substituent; n₁₁₃, n₁₂₃ and n₁₃₃ each independently represent an integer of from 0 to 4; and X⁻ represents a counteranion represented by any of the following Formulae (II) to (IV):

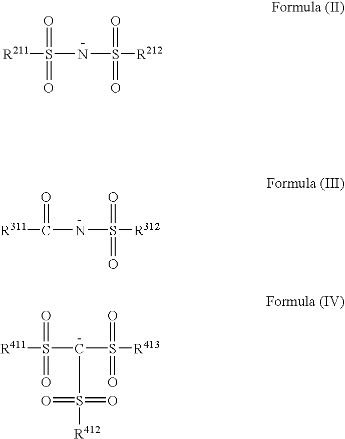

wherein, in Formulae (II) to (IV), R²¹¹, R²¹², R³¹¹, R³¹², R⁴¹¹, R⁴¹² and R⁴¹³ each independently represent an aliphatic group, an aromatic group or a heterocyclic group.

<2>. A triarylamine derivative as described in <1>, wherein in Formulae (II) to (IV), R²¹¹, R²¹², R³¹¹, R³¹², R⁴¹¹, R⁴¹² and R⁴¹³ each independently represent an aliphatic group having a halogen atom, an aromatic group having a halogen atom or a heterocyclic group having a halogen atom.

<3>. A triarylamine derivative as described in <2>, wherein in Formulae (II) to (IV), R²¹¹, R²¹², R³¹¹, R³¹², R⁴¹¹, R⁴¹² and R⁴¹³ each independently represent an aliphatic group having a fluorine atom, an aromatic group having a fluorine atom, or a heterocyclic group having a fluorine atom.

<4>. A triarylamine derivative as described in <1>, wherein, in Formula (I), R¹¹¹, R¹¹², R¹²¹, R¹²², R¹³¹ and R¹³² each independently represent a branched alkyl group.

<5>. A triarylamine derivative as described in <1>, wherein in Formula (I), at least one of R¹¹¹, R¹¹², R¹²¹, R¹²², R¹³¹ and R¹³² represents an alkyl group having a cyano group.

<6>. A triarylamine derivative as described in <1>, wherein in Formula (I), at least one of R¹¹¹, R¹¹², R¹²¹, R¹²², R¹³¹ and R¹³² represents an alkyl group having a halogen atom.

<7>. A triarylamine derivative as described in <6>, wherein the halogen atom is a fluorine atom.

<8>. A triarylamine derivative as described in <1>, wherein n₁₁₃, n₁₂₃, and n₁₃₃ each independently represent 0 or 1.

<9>. A triarylamine derivative as described in <1>, wherein R¹¹¹, R¹¹², R¹²¹, R¹²², R¹³¹ and R¹³² each independently represent a hydrogen atom, an alkyl group, or an aryl group, n₁₁₃, n₁₂₃ and n₁₃₃ each independently represent 0 or 1, R¹¹³, R¹²³ and R¹³³ each independently represent an alkyl group or an alkoxy group, and R²¹¹, R²¹², R³¹¹, R³¹², R⁴¹¹, R⁴¹² and R⁴¹³ each independently represent an alkyl group or an aryl group.

<10>. A triarylamine derivative as described in <1>, wherein R¹¹¹, R¹¹², R¹²¹, R¹²², R¹³¹ and R¹³² each independently represent an alkyl group having a carbon number of from 2 to 6, n₁₁₃, n₁₂₃ and n₁₃₃ each represent 0, and R²¹¹, R²¹², R³¹¹, R³¹², R⁴¹¹, R⁴¹² and R⁴¹³ each independently represent a perfluoroalkyl group having a carbon number of from 1 to 6.

<11>. A triarylamine derivative as described in <1>, wherein R¹¹¹, R¹¹², R¹²¹, R¹²², R¹³¹ and R¹³² each independently represent a branched alkyl group having a carbon number of 2 to 6, n₁₁₃, n₁₂₃ and n₁₃₃ each represent 0, and R²¹¹, R²¹², R³¹¹, R³¹², R⁴¹¹, R⁴¹² and R⁴¹³ each independently represent a perfluoroalkyl group having a carbon number of 1 to 6.

<12>. A triarylamine derivative as described in <1>, wherein at least one of R¹¹¹, R¹¹², R¹²¹, R¹²², R¹³¹ and R¹³² represents an alkyl group having a cyano group and a carbon number of 2 to 6, n₁₁₃, n₁₂₃ and n₁₃₃ each represent 0, and R²¹¹, R²¹², R³¹¹, R³¹², R⁴¹¹, R⁴¹² and R⁴¹³ each independently represent a perfluoroalkyl group having a carbon number of 1 to 6.

<13>. A triarylamine derivative as described in <1>, wherein at least one of R¹¹¹, R¹¹², R¹²¹, R¹²², R¹³¹ and R¹³² represents an alkyl group having a halogen atom and a carbon number of 2 to 6, n₁₁₃, n₁₂₃ and n₁₃₃ each represent 0, and R²¹¹, R²¹², R³¹¹, R³¹², R⁴¹¹, R⁴¹² and R⁴¹³ each independently represent a perfluoroalkyl group having a carbon number of 1 to 6.

<14>. A triarylamine derivative as described in <13>, wherein the halogen atom is a fluorine atom.

### EXAMPLES

The invention will be explained more specifically below by way of Examples, but the present invention is not limited to these Examples. Unless otherwise is indicated, “part” and “%” are based on mass.

### Example 1

### Synthesis of Compound 5

Into 40 ml of DMF was completely dissolved 5 g of tris(4-di(n-butyl)aminophenyl)amine, and the solution was heated and stirred at 60° C. To this was added 15 g of a silver salt of bis(trifluoromethanesulfonyl)imide, and the mixture was heated and stirred at 60° C. for 3 hours. The reaction solution was filtered to remove insoluble components, 200 ml of water was added to the reaction solution, and precipitated crystal was collected by filtration. The resulting crystal was washed with water, and dried to obtain 8.6 g (91%) of a target compound 5.

When a mass spectrum was measured, M⁺ was 627. When an absorption spectrum was measured, a maximum absorption wavelength was λmax=916 nm (dichloromethane).

The results of elemental analysis are shown in Table 5.

### Example 2

### Synthesis of Compound 10

In the same manner as in Example 1 except that tris(4-di(iso-amyl)aminophenyl)amine was used in place of tris(4-di(n-butyl)aminophenyl)amine, 8.5 g (95%) of a target compound 10 was obtained.

When a mass spectrum was measured, M⁺ was 711. When an absorption spectrum was measured, a maximum absorption wavelength was λmax=917 nm (dichloromethane).

The results of elemental analysis are shown in Table 5.

### Example 3

### Synthesis of Compound 14

In the same manner as in Example 1 except that tris(4-bis(3-cyanopropyl)aminophenyl)amine was used in place of tris(4-di(n-butyl)aminophenyl)amine, 8.0 g (89%) of a target compound 14 was obtained.

When a mass spectrum was measured, M⁺ was 693. When an absorption spectrum was measured, a maximum absorption wavelength was λmax=917 nm (dichloromethane).

The results of elemental analysis are shown in Table 5.

### Example 4

### Synthesis of Compound 36

In the same manner as in Example 1 except that a silver salt of trifluoromethanesulfonyl(p-toluenesulfonyl)imide was used in place of a silver salt of bis(trifluoromethanesulfonyl)imide, 9.1 g (93%) of a target compound 36 was obtained.

When a mass spectrum was measured, M⁺ was 627. When an absorption spectrum was measured, a maximum absorption wavelength was λmax=916 nm (dichloromethane).

The results of elemental analysis are shown in Table 5.

### Example 5

### Synthesis of Compound 45

In the same manner as in Example 1 except that a silver salt of trifluoromethanesulfonyl(trifluoroacetyl)imide was used in place of a silver salt of bis(trifluoromethanesulfonyl)imide, 8.0 g (90%) of a target compound 45 was obtained.

When a mass spectrum was measured, M⁺ was 627. When an absorption spectrum was measured, a maximum absorption wavelength was λmax=918 nm (dichloromethane).

The results of elemental analysis are shown in Table 5.

### Example 6

### Synthesis of Compound 50

In the same manner as in Example 2 except that a silver salt of trifluoromethanesulfonyl(trifluoroacetyl)imide was used in place of a silver salt of bis(trifluoromethanesulfonyl)imide, 7.4 g (88%) of a target compound 50 was obtained.

When a mass spectrum was measured, M⁺ was 711. When an absorption spectrum was measured, a maximum absorption wavelength was λmax=917 nm (dichloromethane).

The results of elemental analysis are shown in Table 5.

### Example 7

### Synthesis of Compound 54

In the same manner as in Example 3 except that a silver salt of trifluoromethanesulfonyl(trifluoroacetyl)imide was used in place of a silver salt of bis(trifluoromethanesulfonyl)imide, 7.4 g (87%) of a target compound 54 was obtained.

When a mass spectrum of this compound was measured, M⁺ was 693. When an absorption spectrum was measured, a maximum absorption wavelength was λmax=916 nm (dichloromethane).

The results of elemental analysis are shown in Table 5.

### Example 8

### Synthesis of Compound 76

In the same manner as in Example 4 except that a silver salt of trifluoroacetyl(p-toluenesulfonyl)imide was used in place of a silver salt of trifluoromethanesulfonyl(p-toluenesulfonyl)imide, 8.8 g (95%) of a target compound 76 was obtained.

When a mass spectrum was measured, M⁺ was 627. When an absorption spectrum was measured, a maximum absorption wavelength was λmax=916 nm (dichloromethane).

The results of elemental analysis are shown in Table 5.

### Example 9

### Synthesis of Compound 85

In the same manner as in Example 1 except that a silver salt of tris(trifluoromethanesulfonyl)methyl was used in place of a silver salt of bis(trifluoromethanesulfonyl)imide, 7.0 g (88%) of a target compound 85 was obtained.

When a mass spectrum was measured, M⁺ was 627. When an absorption spectrum was measured, a maximum absorption wavelength was λmax=918 nm (dichloromethane).

The results of elemental analysis are shown in Table 5.

### Example 10

### Synthesis of Compound 90

In the same manner as in Example 2 except that a silver salt of tris(trifluoromethanesulfonyl)methyl was used in place of a silver salt of bis(trifluoromethanesulfonyl)imide in Example 2, 9.9 g (92%) of a target compound 90 was obtained.

When a mass spectrum was measured, M⁺ was 711. When an absorption spectrum was measured, a maximum absorption wavelength was λmax=918 nm (dichloromethane).

The results of elemental analysis are shown in Table 5.

### Example 11

### Synthesis of Compound 94

In the same manner as in Example 3 except that a silver salt of tris(trifluoromethanesulfonyl)methyl was used in place of a silver salt of bis(trifluoromethanesulfonyl)imide in Example 3, 9.8 g (90%) of a target compound 94 was obtained.

When a mass spectrum was measured, M⁺ was 693. When an absorption spectrum was measured, a maximum absorption wavelength was λmax=917 nm (dichloromethane).

The results of elemental analysis are shown in Table 5.

TABLE 5

| | | Theoretical value | Measured value |
|---|---|---|---|
| Example 1 | Compound 5 | C, 46.53%; H, 5.60%; | C, 46.50%; H, 5.65%; N, 7.05% |
| | | N, 7.08% | |
| Example 2 | Compound | C, 49.12%; H, 6.18%; | C, 49.10%; H, 6.21%; N, 6.58% |
| | 10 | N, 6.61% | |
| Example 3 | Compound | C, 44.09%; H, 3.86%; | C, 44.06%; H, 3.89%; N, |
| | 14 | N, 13.41% | 13.38% |
| Example 4 | Compound | C, 52.01%; H, 5.57%; | C, 51.97%; H, 5.61%; N, 6.24% |
| | 36 | N, 6.27% | |
| Example 5 | Compound | C, 51.70%; H, 5.97%; | C, 51.69%; H, 5.99%; N, 7.51% |
| | 45 | N, 7.54% | |
| Example 6 | Compound | C, 54.08%; H, 6.56%: | C, 54.05%; H, 6.59%; N, 7.00% |
| | 50 | N, 7.01% | |
| Example 7 | Compound | C, 48.81%; H, 4.10%; | C, 48.79%; H, 4.13%; |
| | 54 | N, 14.23% | N, 14.20% |
| Example 8 | Compound | C, 52.01%; H, 5.57%; | C, 52.00%; H, 5.61%; N, 6.23% |
| | 76 | N, 6.27% | |
| Example 9 | Compound | C, 39.67%; H, 4.58%; | C, 39.64%; H, 4.61%; N, 5.76% |
| | 85 | N, 5.78% | |
| Example 10 | Compound | C, 42.18%; H, 5.11%; | C, 42.14%; H, 5.14%; N, 5.44% |
| | 90 | N, 5.47% | |
| Example 11 | Compound | C, 37.95%; H, 3.18%; | C, 37.92%; H, 3.21%; |
| | 94 | N, 11.06% | N, 11.03% |

<Assessment>

The triarylamine derivatives according to the invention obtained above, and triarylamine derivatives of Comparative Examples shown in the following in Table 6 were subjected to the following assessment.

### (Sample for Assessment)

2.5 g of PMMA and 0.125 g of the triarylamine derivative were dissolved in 25 ml of dichloromethane, and the solution was coated on a 100 μm-thick PET film. This was dried at 80° C. for 2 hours under reduced pressure to prepare a sample for assessment having a thickness of from 2 to 3 μm. The sample for assessment contained the triarylamine derivative at 5% by weight based on PMMA.

(Heat Resistance)

The sample for assessment obtained above was heat-treated at 80° C. for 400 hours. The absorbance of the assessment sample at the maximum absorption wavelength of the triarylamine derivative (colorant) was measured, and a colorant residual rate (%) was obtained as a ratio of the absorbance after heat treatment relative to the absorbance before heat treatment. The results are shown in Table 6.

(Wet Heat Resistance)

The sample for assessment obtained above was subjected to wet heat treatment at 60° C. and a relative humidity of 90% for 200 hours. The absorbance of the assessment sample at the maximum absorption wavelength of the triarylamine derivative (colorant) was measured, and a colorant residual rate (%) was obtained as a ratio of the absorbance after wet heat treatment relative to the absorbance before wet heat treatment. The results are shown in Table 6.

(Light Resistance)

The sample for assessment obtained above was irradiation-treated for 170 hours with a xenon lamp (170,000 lux, using an UV cut filter). The absorbance of the assessment sample at the maximum absorption wavelength of the triarylamine derivative (colorant) was measured, and a colorant residual rate (%) was obtained as a ratio of the absorbance after irradiation treatment relative to the absorbance before irradiation treatment. The results are shown in Table 6.

(Solubility)

Solubility (w/v %) of the triarylamine derivative in 2-butanone (MEK) was measured. The results are shown in Table 6.

TABLE 6

| | Heat | Wet Heat | Light | Solubility | |
|---|---|---|---|---|---|
| Triarylamine Derivative | Resistance | Resistance | Resistance | (w/v) | Remarks |
| 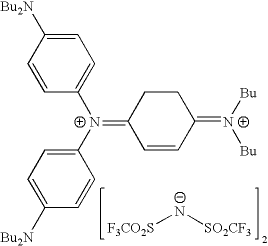 | 83% | 85% | 99% | 27% | Example Compound 5 |
| 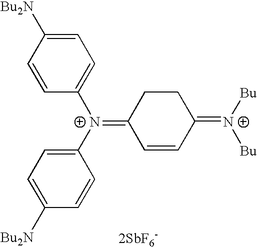 | 80% | 45% | 98% | 16% | Comparative Example |
| 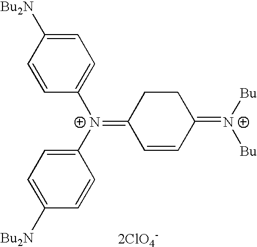 | 0% | 19% | 80% | 15% | Comparative Example |

From Table 6, it is seen that the triarylamine derivative according to the invention is a near infrared absorbing triarylamine derivative which is excellent in heat resistance, light resistance and solubility.

According to the present invention, a near infrared absorbing triarylamine derivative can be provided which has higher safety, and is good in heat resistance and solubility.

All publications, patent applications, and technical standards mentioned in this specification are herein incorporated by reference to the same extent as if each individual publication, patent application, or technical standard was specifically and individually indicated to be incorporated by reference.

What is claimed:

1. A triarylamine derivative represented by the following Formula (I): 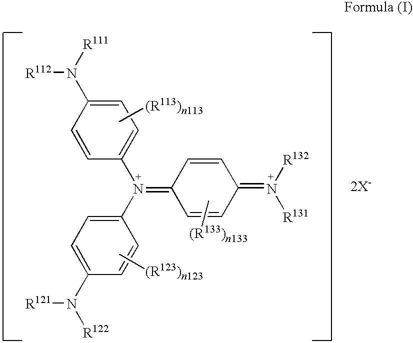
wherein, in Formula (I): R¹¹¹, R¹¹², R¹²¹, R¹²², R¹³¹ and R¹³² each independently represent a hydrogen atom, an aliphatic group or an aromatic group, provided that R¹³¹ and R¹³² are not both phenyl groups having a group represented by —NR¹⁴¹R¹⁴² at a fourth position; R¹⁴¹ and R¹⁴² each independently represent a hydrogen atom, an aliphatic group, or an aromatic group; R¹¹³, R¹²³ and R¹³³ each independently represent a substituent selected from the group consisting of a halogen, an alkyl group, an alkenyl group, an alkynyl group, an aryl group, a heterocyclic group, a cyano group, a hydroxyl group, a nitro group, a carboxyl group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl- or aryl-sulfonylamino group, a mercapto group, an alkylthio group, an arylthio group, a heterocyclic thio group, a sulfamoyl group, a sulfo group, an alkyl- or aryl-sulfinyl group, an alkyl- or aryl-sulfonyl group, an acyl group, an aryloxycarbonyl group, an alkoxycarbonyl group, a carbamoyl group, an aryl-azo group, a heterocyclic-azo group, an imido group, a phosphino group, a phosphinyl group, a phosphinyloxy group, a phosphinylamino group, and a silyl group; n₁₁₃, n₁₂₃ and n₁₃₃ each independently represent an integer of from 1 to 4; and X⁻ represents a counteranion represented by any of the following Formulae (II) to (IV): 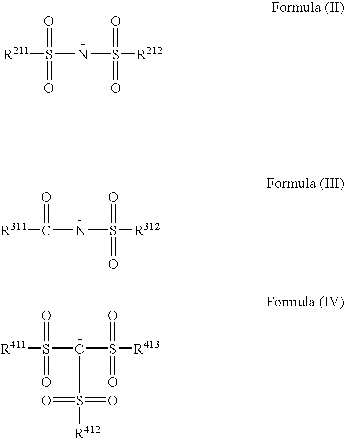
wherein, in Formulae (II) to (IV), R²¹¹, R²¹², R³¹¹, R³¹², R⁴¹¹, R⁴¹² and R⁴¹³ each independently represent an aliphatic group, an aromatic group or a heterocyclic group.

2. The triarylamine derivative according to claim 1, wherein in Formulae (II) to (IV), R²¹¹, R²¹², R³¹¹, R³¹², R⁴¹¹, R⁴¹² and R⁴¹³ each independently represent an aliphatic group having a halogen atom, an aromatic group having a halogen atom or a heterocyclic group having a halogen atom.

3. The triarylamine derivative according to claim 2, wherein in Formulae (II) to (IV), R²¹¹, R²¹², R³¹¹, R³¹², R⁴¹¹, R⁴¹² and R⁴¹³ each independently represent an aliphatic group having a fluorine atom, an aromatic group having a fluorine atom, or a heterocyclic group having a fluorine atom.

4. The triarylamine derivative according to claim 1, wherein, in Formula (I), R¹¹¹, R¹¹², R¹²¹, R¹²², R¹³¹ and R¹³² each independently represent a branched alkyl group.

5. The triarylamine derivative according to claim 1, wherein in Formula (I), at least one of R¹¹¹, R¹¹², R¹²¹, R¹²², R¹³¹ and R¹³² represents an alkyl group having a cyano group.

6. The triarylamine derivative according to claim 1, wherein in Formula (I), at least one of R¹¹¹, R¹¹², R¹²¹, R¹²², R¹³¹ and R¹³² represents an alkyl group having a halogen atom.

7. The triarylamine derivative according to claim 6, wherein the halogen atom is a fluorine atom.

8. The triarylamine derivative according to claim 1, wherein n₁₁₃, n₁₂₃, and n₁₃₃ each independently represent 1.

9. The triarylamine derivative according to claim 1, wherein R¹¹¹, R¹¹², R¹²¹, R¹²², R¹³¹ and R¹³² each independently represent a hydrogen atom, an alkyl group, or an aryl group, n₁₁₃, n₁₂₃ and n₁₃₃ each independently represent 1, R¹¹³, R¹²³ and R¹³³ each independently represent an alkyl group or an alkoxy group, and R²¹¹, R²¹², R³¹¹, R³¹², R⁴¹¹, R⁴¹² and R⁴¹³ each independently represent an alkyl group or an aryl group.

10. The triarylamine derivative according to claim 1, wherein, in Formula (I), R¹¹³, R¹²³, and R¹³³ each independently represent a halogen atom, an alkyl group, an alkenyl group, an alkynyl group, an aryl group, a cyano group, a hydroxyl group, a carboxyl group, an alkoxy group, an aryloxy group, a silyloxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl- or aryl-sulfonylamino group, a mercapto group, an alkylthio group, an arylthio group, a sulfamoyl group, a sulfo group, an alkyl- or aryl-sulfinyl group, an alkyl- or aryl-sulfonyl group, an acyl group, an aryloxycarbonyl group, an alkoxycarbonyl group, carbamoyl group, an imido group, a phosphino group, a phosphinyl group, a phosphinyloxy group, a phosphinylamino group or a silyl group.

11. The triarylamine derivative according to claim 1, wherein, in Formula (I), R¹¹³, R¹²³, and R¹³³ each independently represent a halogen atom, an alkyl group, an alkynyl group, an aryl group, a cyano group, a hydroxyl group, a carboxyl group, an alkoxy group, an aryloxy group, a silyloxy group, an amino group, an alkylthio group, an arylthio group, an imido group, or a silyl group.

12. The triarylamine derivative according to claim 1, wherein, in Formula (I), R¹¹³, R¹²³, and R¹³³ each independently represent a halogen atom, an alkyl group, an aryl group, an alkoxy group, an aryloxy group, a silyloxy group or an amino group.

13. The triarylamine derivative according to claim 1, wherein, in Formula (I), R¹¹³, R¹²³, and R¹³³ each independently represent an alkyl group or an alkoxy group.

14. The triarylamine derivative according to claim 1, wherein, in Formula (I), all of R¹¹³, R¹²³, and R¹³³ are the same substituent.

15. The triarylamine derivative according to claim 1, wherein, in Formula (I), X⁻ represents a counteranion represented by Formula (III).
